(19) United States
(12) Patent Application Publication
(10) Pub. No.: US 2024/0058298 A1
(43) Pub. Date: Feb. 22, 2024

(54) GENISTEIN FOR USE IN THE TREATMENT OF FOCAL SEGMENTAL GLOMERULOSCLEROSIS (FSGS)

(71) Applicant: Delta 4 GmbH, Vienna (AT)

(72) Inventor: Paul Perco, Vienna (AT)

(21) Appl. No.: 18/265,192

(22) PCT Filed: Dec. 3, 2021
(86) PCT No.: PCT/EP2021/084252

§ 371 Date: Jun. 2, 2023

(30) Foreign Application Priority Data
Dec. 3, 2020 (EP) 20211547.3

(51) Int. Cl. A61K 31/352; A61P 13/12

(52) U.S. Cl. CPC A61K 31/352; A61P 13/12

(57) ABSTRACT

The present invention relates to a pharmaceutical preparation comprising genistein, or a salt or solvate thereof, for use in the treatment of focal segmental glomerulosclerosis (FSGS).

### FIELD OF THE INVENTION

The present invention relates to a pharmaceutical preparation comprising genistein, or a salt or solvate thereof, for use in the treatment of focal segmental glomerulosclerosis (FSGS).

### BACKGROUND OF THE INVENTION

Focal segmental glomerulosclerosis (FSGS) is a severe glomerulopathy frequently leading to end stage kidney disease. FSGS is used to describe a histopathological lesion defined by the presence of sclerosis in parts (segmental) or some glomeruli (focal). This lesion can be both a disease characterized by primary podocyte injury, or secondary to other impacts including hypertension, obesity or viruses. FSGS can be found without an identifiable cause (idiopathic).

The most common manifestation of FSGS is proteinuria ranging from subnephrotic to nephrotic levels (heavy proteinuria, hypoalbuminemia and hyperlipidemia). Heavy proteinuria leads to progressive loss of kidney function (glomerulosclerosis) and kidney failure. It accounts for about 15% of end-stage renal disease (ESRD). Massive proteinuria (>10-15 g/day) leads to rapid deterioration of renal function and progression to ESRD within 2-3 years. The survival rate of FSGS patients with massive proteinuria is only 45%.

There is no approved drug for FSGS. The current standard of care for patients with FSGS include steroids, ACE inhibitors or ARBs, immunosuppressive drugs such as glucocorticoids followed by calcineurin inhibitors, if needed, for intolerance or inadequate response to glucocorticoids; diuretics, plasmapheresis, diet change and statins.

There are, however, only poor response rates. Renin-angiotensin-aldosterone (RAAS) blockers are also used to control proteinuria, an important signature of FSGS. Existing treatments, however, achieved only limited success.

About 5400 patients are diagnosed with FSGS every year in the United States, but the number of cases is rising more than any other cause of Nephrotic Syndrome. Approximately 1,000 FSGS patients receive kidney transplants every year. Within hours to weeks after a kidney transplant, however, FSGS returns in approximately 30-40% of patients. Only 20% of patients, however, achieve complete remission after 5 years of treatment, and 40% of patients show no remission (Troyanov S. et al., 2005).

WO20200160225A1 refers to artificial estrogen receptor l agonists for inhibition of fibrotic conditions.

Yousefinejad A. et. al. (2019) studied the effect of genistein, L-carnitine and their combination of gene expression of hepatocyte PPAR-α and CPT-1 in experimental nephrotic syndrome. Thereby, the effect of genistein and genistein in combination with L-carnitine on the urine protein and urine protein-to-creatinine ratio was studied. Synergistic effects of L-carnitine and genistein in experimental nephrotic syndrome are shown.

Javanbakht M. H. et. al. (2014) studied the effect of genistein in combination with soy protein on the antioxidant status of kidney tissue in experimental nephrotic syndrome.

Jia Q. et. al. (2018) studied the antifibrogenic effects of genistein on the kidney in streptozotocin-induced diabetic rats and it was shown that genistein exhibited reno-protective effects in diabetic rats.

Despite best care, treatment failure is common and FSGS is causal in a significant proportion of end stage renal disease. Thus, an unmet need exists for novel disease modifying treatments for FSGS.

### SUMMARY OF THE INVENTION

The objective is solved by the subject of the present claims and as further described herein. It had been surprisingly found that the compound of the present invention significantly lowered urine protein/creatinine ratio (UPCR) and urine albumin/creatinine ratio (UACR) in animal studies thus demonstrating significant functional improvement and reduced proteinuria.

The invention provides a pharmaceutical preparation comprising genistein, or a salt or solvate thereof, for use in the treatment of focal segmental glomerulosclerosis (FSGS).

Specifically, genistein can be present in the pharmaceutical preparation as single active agent.

Specifically, FSGS is a recurrent focal segmental glomerulosclerosis, primary FSGS or native FSGS, specifically it is a glomerulopathy characterized by any one of heavy proteinuria, nephrotic syndrome, glomerulonephritis, membranoproliferative glomerulonephritis, IgA glomerulonephritis, progressive renal failure, or glomerular lesions on histopathology.

More specifically, the FSGS is primary FSGS

According to an embodiment of the invention, the pharmaceutical preparation is a medicinal product or a drug product, comprising genistein, and a pharmaceutically acceptable carrier.

Specifically, the pharmaceutical preparation described herein is administered systemically.

According to a further embodiment, genistein used in the preparation described herein comprises the structure

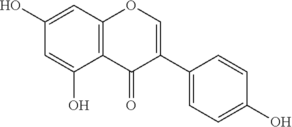 [SMILES: O=c1c(-c2ccc(O)cc2)coc2cc(O)cc(O)c12]

Specifically, the preparation comprises about 10 to 100 g genistein.

According to a further embodiment, pharmaceutical preparation described herein is formulated for systemic administration, preferably for intravenous, intramuscular, subcutaneous, intradermal, transdermal, or oral administration.

According to a further embodiment, the pharmaceutical preparation described herein is administered to the subject as a spray, a powder, a gel, an ointment, a cream, a foam, or a liquid solution, a lotion, a gargle solution, an aerosolized powder, an aerosolized liquid formulation, granules, capsules, drops, tablet, syrup, lozenge, or a preparation for infusion or injection.

According to the invention the pharmaceutical preparation is for use in a subject is treated who suffers from FSGS or is at risk of developing FSGS.

In a further embodiment, the pharmaceutical preparation described herein is applied in an effective amount into a subject suffering from FSGS or being at risk of developing FSGS.

Specifically, the preparation is administered as the sole substance, or wherein treatment or preparation is combined with a further treatment or preparation with one or more active substances.

More specifically, the preparation is administered in combination with an active agent selected from the group consisting of antiviral drugs, anticoagulants, immune modulators, antibody preparations from human sources, monoclonal antibodies, intensive care medications, antihypertensive agents, statins, vasodilators, steroids, cytotoxic drugs, diuretics, non-steroidal anti-inflammatory drugs (NSAIDs), cholesterol or triglyceride reducing agents.

### FIGURES

FIG. 1: FSGS was induced with Adriamycin (ADR) and evaluated regarding proteinuria at the indicated times (in days) after induction. Box plots and p-values of all individual collected urines are shown. Repeated measures ANOVA revealed a significant beneficial impact of genistein treatment over time on outcome determined based on UPCR levels UPCR=urinary protein creatinine; boxes marked “ctrl”=control mice with FSGS; boxes marked “Genistein”=mice with FSGS, treated with Genistein; Boxplots indicate the median (midline), inter-quartile range (box) and 95% confidence intervals (whiskers) in addition to individual data points (scatter).

### DETAILED DESCRIPTION

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−10% or +/−5% of the given value.

Genistein as described herein may be used as a “physiologically acceptable salt”. The choice of salt is determined primarily by how acid or basic the chemical is (the pH), the safety of the ionized form, the intended use of the drug, how the drug is given (for example, by mouth, injection, or on the skin), and the type of dosage form (such as tablet, capsule, or liquid).

Exemplary salts which are physiologically acceptable are sodium salts. However, it is also possible to employ, in place of the sodium salts, other physiologically acceptable salts, e.g., other alkali metal salts, alkaline earth metal salts, ammonium salts and substituted ammonium salts. Specific examples are potassium, lithium, calcium, aluminum and iron salts. Preferred substituted ammonium salts are those derived, for example, from lower mono-, di-, or trialkylamines, or mono-, di- and trialkanolamines. The free amino acids per se can also be used. Specific examples are ethylamine, ethylenediamine, diethylamine, or triethylamine salts.

The term “subject” or “patient” as used herein refers to a human being, or a non-human mammal, such as a dog, cat, horse, camelids, cattle or pig, suffering from FSGS or being at risk of developing FSGS. Specifically, it is a human being.

The term “non-diabetic subject” refers to a subject not suffering from Diabetes mellitus.

A subject “at risk” of developing FSGS may or may not have detectable disease or symptoms of disease, and may or may not have displayed detectable disease or symptoms of disease prior to the treatment methods described herein. “At risk” denotes that a subject has one or more risk factors, which are measurable parameters that correlate with development of FSGS, as described herein and known in the art. A subject having one or more of these risk factors has a higher probability of developing FSGS than a subject without one or more of these risk factor(s).

A “subject” can be a “patient”. A “patient,” refers to a “subject” who is under the care of a treating physician. In another embodiment, the patient is a subject who has not been diagnosed with FSGS. In yet other embodiments, the patient is a subject who has been diagnosed with FSGS but has not had any treatment to address the FSGS.

The term “pharmaceutically acceptable” also referred to as “pharmacologically acceptable” means compatible with the treatment of animals, in particular, humans. The term pharmacologically acceptable salt also includes both pharmacologically acceptable acid addition salts and pharmacologically acceptable basic addition salts.

The term “pharmacologically acceptable acid addition salt” as used herein means any non-toxic organic or inorganic salt of any base compound of the disclosure, or any of its intermediates. Basic compounds of the disclosure that may form an acid addition salt include, for example, compounds that contain a basic nitrogen atom. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either mono-, di- or triacid salts can be formed, and such salts may exist in either a hydrated, solvated or substantially anhydrous form. In general, the acid addition salts of the compounds of the disclosure are more soluble in water and various hydrophilic organic solvents, and generally demonstrate higher melting points in comparison to their free base forms. The selection of the appropriate salt will be known to one skilled in the art. Other non-pharmacologically acceptable acid addition salts, e.g. oxalates, may be used, for example, in the isolation of the compounds of the disclosure, for laboratory use, or for subsequent conversion to a pharmacologically acceptable acid addition salt.

Specifically, genistein may be present as genistein sodium salt dihydrate, genistein potassium salt dihydrate, or genistein calcium salt dihydrate, etc.

Genistein may also be present as crystalline form including crystalline genistein sodium salt dihydrate; crystalline genistein potassium salt dihydrate; crystalline genistein calcium salt; crystalline genistein magnesium salt; crystalline genistein L-lysine salt; crystalline genistein N-methylglucamine salt; crystalline genistein N-ethylglucamine salt; crystalline genistein diethylamine salt; and crystalline genistein monohydrate as described in US20150290166.

Genistein (5,7-Dihydroxy-3-(4-hydroxyphenyl)chromen-4-one, 5,7-Dihydroxy-3-(4-hydroxyphenyl)-4H-1-benzopyran-4-one, 4′,5,7-Trihydroxyisoflavone) comprises the structure

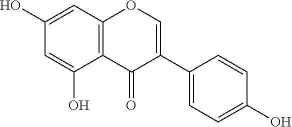 [SMILES: O=c1c(-c2ccc(O)cc2)coc2cc(O)cc(O)c12]

Genistein is sold under the trade names BIO 3000, Baichanin A, Bonistein, Climagen F, Genivida, Prunetol, Differenol A.

Genistein is an isoflavonoid derived from soy products. It inhibits protein-tyrosine kinase and topoisomerase-II (DNA topoisomerases, type II) activity and is in clinical trials as antineoplastic and antitumor agent. Experimentally, it has been shown to induce G2 phase arrest in human and murine cell lines. Additionally, genistein has antihelmintic activity. It has been determined to be the active ingredient in Felmingia vestita, which is a plant traditionally used against worms. It has shown to be effective in the treatment of common liver fluke, pork trematode and poultry cestode.

Furthermore, genistein is a phytoestrogen and selective estrogen receptor modulator. It has been investigated in clinical trials as an alternative to classical hormone therapy to help prevent cardiovascular disease in postmenopausal women.

Genistein can stimulate autophagy in podocytes (Wang Y. et al., 2018) and modulate renal NFκB-dependent inflammation and podocyte abnormalities (Palanisamy N. et al., 2011). The natural compound genistein can act as PPAR-gamma agonist (Song M. et al., 2015; Zhang T. et al, 2013) and was shown to influence additional pathways relevant for FSGS development and/or progression. Genistein was shown to inhibit proteins involved in inflammation such as JAK2, TLR3, TLR4, or STAT3 (Gholampour F. et al., 2020; Ma W. et al., 2015; Zhou X. et al., 2014; Cheng W.X. et al., 2019).

Genistein is also capable of modulating expression of genes involved in fibrotic processes and extracellular matrix dysregulations such as MMP2, MMP14, or FN1 (Sundaram M. K. et al., 2019; Yazdani Y. et al., 2016, Tong M. et al., 2012). Genistein can also reduce levels of PLAU (Santibariez J. F. et al., 2000), a molecule involved in the coagulation cascade being investigated as biomarker in chronic kidney disease.

Genistein also reduces levels of SPP1 (Mentor-Marcel R. et al., 2005), a molecule highly upregulated in FSGS. Genistein modulates activation of HMOX1 and HIF1A, two proteins that are linked to renoprotection.

Genistein comprises about 10 to 100 mg per dose. Specifically, it can comprise 20 10, 20, 30, 40, 50, 60, 70, 75, 80, 90, 100 mg genistein. It may be administered continuously at the same dose or as bolar administration at high dose, such as about and further as low or reduced maintenance dose.

FSGS is a distinct clinico-pathological medical condition characterized by focal and segmental sclerosis in the kidney glomerulus and by podocyte foot process effacement (D'Agati V., 2003; Rosenberg A. Z. and Kopp J. B., 2017, Bose B. and Cattran D., 2014). The pathophysiology of FSGS can origin within the glomerulus (“primary FSGS”) or be secondary to other reasons (e.g. hypertension; D′Agati V., 2003, Rosenberg A. Z. and Kopp J. B., 2017, Bose B. and Cattran D., 2014). Primary FSGS frequently leads to nephrotic syndrome characterised by proteinuria, hypoalbuminuria, hyperlipidemia and edema (D'Agati V., 2003; Rosenberg A. Z. and Kopp J. B., 2017, Bose B. and Cattran D., 2014). For a high number of cases the exact cause of FSGS is still elusive (idiopathic; D'Agati V., 2003; Rosenberg A. Z. and Kopp J. B., 2017, Bose B. and Cattran D., 2014). Known etiologies for FSGS are heterogeneous and include gene mutations, drugs, viruses, hypertension, and circulating factors—but not by diabetes (D'Agati V., 2003; Rosenberg A. Z. and Kopp J. B., 2017, Bose B. and Cattran D., 2014). Diabetic nephropathy—including diabetic glomerulosclerosis—represents the chronic loss of kidney function as a result of the pathophysiology of Diabetes mellitus (Qi et al., 2017).

Podocytes are atypical epithelial cells in the Bowman capsule in kidneys that wrap around capillaries of the glomerulus.

As used herein, FSGS is a scarring of glomeruli (sclerosis) and a damage to podocytes, specifically triggered by an endogenous factor that recognizes the podocyte as its target (podocytopathy, idiopathic or primary FSGS), or caused by an identifiable cause that indirectly injures podocytes (secondary FSGS), such as a genetic disorder, hypertension, inflammation, deregulation of proliferation, or mechanical stress.

The distinctive morphological picture of primary FSGS is a diffuse podocytopathy (+at least one FSGS lesion) clinically dominated by a nephrotic syndrome, The diffuse podocyte toxicity involves almost all podocytes, with more than 50% of foot process effacement at the electron microscopy. A massive loss of albumin in the urine as well as hypoalbuminemia are commonly observed but are unusual in secondary forms even if nephrotic range proteinuria is present. Microvillous transformation, cytoplasm shedding, increased density of actin cytoskeleton on effaced foot processes, increased number of lysosomes and auto-phagocytic bodies are common features. Due to structural reorganization the risk of detachment of podocytes from the glomerular basement membrane (GBM) is increased, leaving wide areas of bare GBM. These “sticky” areas can produce adhesions, after which the deposition of hyaline material and mesangial matrix expansion progressively narrows the capillary lumen, until the affected segment is obstructed and sclerosed. Reversible glomerular prolapses into the proximal tubule are occasionally observed, and are the expression of an acute enlargement of the tuft and predictive of a ‘tip lesion’ (Angioi A., and Pani, A., 2016).

Genetic forms of FSGS show similar alterations as described for primary FSGS.

Blood hypertension and adaptive mechanism can be major causes for secondary FSGS, both causing glomerular hypertension, accompanied by hypertrophied and stretched podocytes. Due to mechanical stress on the podocyte anchorage, the foot processes, structural reorganization of the cytoskeleton is generated, causing focal effacement.

The most aggressive form of FSGS is collapsing glomerulopathy, histologically shown by foot process effacement, segmental to global collapse of the capillary tuft, surrounded by a crown of hyperplastic podocytes (Angioi A., and Pani, A., 2016).

The symptoms of FSGS are also heavy proteinuria with optional biopsy confirmation of FSGS with glomerulosclerosis, glomerulonephritis (e.g. membranoproliferative glomerulonephritis (MPGN), IgA glomerulonephritis), nephrotic syndrome (hypoalbuminuria, hyperlipidemia, edema), progressive renal failure, glomerular lesions on histopathology, specifically as classified according to Haas M. et al., 2013, and podocyte fusion and injury.

FSGS can be diagnosed by methods well known in the art, such as, but not limited to determination of urinary protein/creatinine ratio (UPCR), urinary albumin/creatinine ratio (UACR), light microscopy of kidney biopsy, e.g. glomerular size, histologic variant of FSGS, microcystic tubular changes, and tubular hypertrophy; immunofluorescence, e.g. to rule out other primary glomerulopathies; and electron microscopy, e.g. extent of podocyte foot process effacement, podocyte microvillous transformation, and tubuloreticular inclusions. Specifically, FSGS refers to non-diabetic renal disease. Specifically, diabetic nephropathy caused by Diabetes mellitus is excluded from the use of genistein for the treatment of FSGS. Non-diabetic renal disease and diabetic renal disease can be confirmed and distinguished by methods known in the art such as biopsy.

In diabetes patients, nodular lesions and diffuse lesions are often observed. Classification methods for diabetic nephropathies are well known by the skilled person and are described by Qi C. et al., 2017, Tervaert T.W.C. et al., 2010, and Fioretto P. et al., 1996. Fioretto classification includes tubular, interstitial, and vascular lesions and divided diabetic nephropathies into 3 categories according to the pathological changes under light microscope: C1, normal/near normal; C2, typical diabetic nephropathy with predominantly glomerular changes; and C3, atypical patterns of injury, associated with disproportionately damage including tubulointerstitial or arteriolar hyalinosis and with absent or only mild diabetic glomerular changes. Tervaert pathological classification divides diabetic nephropathies into four classifications according to glomerular lesions, along with a separate scoring system for tubular, interstitial, and vascular lesions. Specifically, patients suffering from diabetes, specifically patients who are suffering from diabetes for more than 2 years, are excluded from being treated by genistein as described herein.

Diabetic related nephropathies include diabetic retinopathy, diabetic nephropathy, and diabetes-related glomerulosclerosis.

The use of the term “FSGS” covers native FSGS, primary FSGS as well as recurrent FSGS, but specifically excluding diabetic FSGS,

Glomerulonephritis describes the inflammation of the membrane tissue in the kidney that serves as a filter, separating wastes and extra fluid from the blood.

MPGN is a form of glomerulonephritis caused by an abnormal immune response. Deposits of antibodies build up in a part of the kidneys called the glomerular basement membrane.

Glomerulosclerosis describes the scarring or hardening of the tiny blood vessels within the kidney. Although glomerulonephritis and glomerulosclerosis have different causes, they can both lead to kidney failure.

Nephrotic syndrome is a kidney disorder that causes the body to pass too much protein in urine. Nephrotic syndrome is usually caused by damage to the clusters of small blood vessels in kidneys.

As used herein, recurrent FSGS (rFSGS), or recurrence of FSGS is defined by heavy proteinuria with optional biopsy confirmation of FSGS with glomerular sclerosis and podocyte fusion and injury without evidence of acute rejection, glomerulitis or allograft glomerulopathy. As used herein, a recurrent FSGS (rFSGS) subject or patient is defined as someone who had FSGS prior to kidney transplant and then developed a recurrence of FSGS (rFSGS) following kidney transplant.

As used herein, a non-recurrent FSGS (nrFSGS) subject or patient is defined as someone has FSGS prior to kidney transplant but does not develop FSGS following kidney transplant.

As used herein, a native FSGS (nFSGS) subject or patient is defined as someone who has FSGS (heavy proteinuria with optional biopsy confirmation of FSGS with glomerular sclerosis and podocyte fusion and injury) in its own kidney prior to transplant.

The pharmaceutical preparation can be a medicinal product or a drug product, comprising genistein and a pharmaceutically acceptable carrier. The preparation described herein can also be a nutraceutical or supplement composition, specifically used as food supplement.

The term “nutraceutical” as used herein denotes usefulness in both the nutritional and pharmaceutical field.

Herein provided is also a method for treatment of FSGS wherein the pharmaceutical preparation comprises genistein in an effective amount.

Specifically, Genistein or a pharmaceutically acceptable salt or solvate thereof significantly reduces podocyte injury and podocytopathy in FSGS.

The term “effective amount” or “pharmaceutically effective amount” refers to that amount of compound that produces the desired effect for which it is administered (e.g., improvement in symptoms of FSGS, lessening the severity of FSGS or a symptom of FSGS, and/or reducing progression of FSGS or a symptom of FSGS). The exact amount of an effective dose will depend on the purpose of the treatment and will be ascertainable by one skilled in the art using known techniques (see, e.g., Lloyd V. A., 2016).

As used herein, the term “treatment” and its cognates refer to slowing or stopping disease progression. “Treatment” and its cognates as used herein, include, but are not limited to the following: complete or partial remission, lower risk of kidney failure (e.g. ESRD), and disease-related complications (e.g. edema, susceptibility to infections, or thrombo-embolic events). Improvements in or lessening the severity of any of these symptoms can be readily assessed according to methods and techniques known in the art or subsequently developed. Desirable effects of treatment include preventing the occurrence or recurrence of FSGS or a condition or symptom thereof, alleviating a condition or symptom of FSGS, diminishing any direct or indirect pathological consequences of FSGS, decreasing the rate of FSGS progression or severity, and/or ameliorating or palliating the FSGS. In some embodiments, methods and compositions of the invention are used on patient sub-populations identified to be at risk of developing FSGS. In some cases, the methods and compositions of the invention are useful in attempts to delay development of FSGS.

In some embodiments, the compound of the pharmaceutical preparation described herein (i.e. genistein), or a pharmaceutically acceptable salt or solvate thereof is not administered with any other therapeutic compound. In some embodiments, the compound is not administered with any other therapeutic compound, concurrently or sequentially. In some embodiments, the compound is administered alone. In some embodiments, the method further comprises administering to the patient one or more additional therapeutic compound. In some embodiments, the one or more additional therapeutic compound is selected from one or more of an antiviral drug, an anticoagulant, an immune modulator, an antibody preparation from human sources, a monoclonal antibody, an intensive care medication, an antihypertensive agent, a statin, a vasodilator, an steroid, a cytotoxic drug, a diuretic, a non-steroidal anti-inflammatory drug (NSAID), a cholesterol or triglyceride reducing agent, PPAR agonist or a phytopharmacological compound.

The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 22nd edition, 2013, Pharmaceutical Press, and Encyclopedia of Pharmaceutical Technology, 2004, Taylor & Francis, disclose various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical preparation, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate, microcrystalline cellulose, methyl cellulose, sodium carboxymethyl cellulose), lactose, dextrin, mannitol, white sugar, maize starch, pre-gelatinized starch, precipitated calcium carbonate and calcium hydrogen phosphate, powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants or any mixtures and combinations thereof.

### EXAMPLES

The foregoing description will be more fully understood with reference to the following examples. Such examples are, however, merely representative of methods of practicing one or more embodiments of the present invention and should not be read as limiting the scope of invention.

### Example 1

In vivo Testing

For biological testing of genistein, the accepted mouse models of FSGS, was chosen, i.e. the adriamycin induced nephropathy. Adriamycin nephropathy is the rodent state-of-the-art model of human primary focal segmental glomerulosclerosis (FSGS) and is characterized by podocyte injury followed by glomerulosclerosis, tubulointerstitial inflammation and fibrosis (Da Sacco S. et al., 2014, Lee V. W. and Harris D. C., 2011) and was therefore used for demonstrating the therapeutic effect of genistein in FSGS. All animal experiments and handling were in accordance with the Austrian law for protection of animals and approved by the animal ethics committee of the Austrian ministry for science and research.

Drugs and Chemicals

Adriamycin (Doxorubicin; Sigma, D1515-10 mg) was diluted in ddH₂O. To induce FSGS, a dose of 10.5 mg/kg mouse was injected i.v. into the tail vein using 27G needles, i.e. a total volume of 200 μl volume per 25 g mouse.

For tail vein injection, mice were anaesthetized using Ketanest (Pfizer, 25 mg/ml vials) and Rompun (Bayer, 20 mg/ml injection solution). A final mix of 12.5 mg/ml Ketanest and 0.25% Rompun in 0.9% NaCl solution was prepared and 200 μl/25 g mouse were injected. Genistein (Selleckchem, S1342) was used for therapy experiments.

### Mice and FSGS Model

Female Balb/c mice were obtained from the animal breeding facility of the Medical University (Abteilung kw Labortierkunde, Himberg, Austria). 8-18 weeks old animals with a weight >17g were used for the experiments and adjusted for weight and age. Animals were allowed acclimatization of at least 7 days. Animal were marked by ear or toe clip codes to allow identification. Adriamycin nephropathy in Balb/c mice is the most commonly used animal model for preclinical FSGS studies and shares the central features with human FSGS.

Standard commercial chow from ssniff (ssniff Spezialdiäten GmbH, Soest, Germany) contained crude protein (19%), crude fat (3.3%), crude fiber (4.9%), crude ash (6.4%), starch (36.5%), sugar (4.7%). Control group animals were fed standard chow, test group animals standard chow+genistein (500 mg/kg) produced by ssniff. All animals were allowed to eat ad libitum assuming a daily average uptake of about 4 grams/mouse (according to animal care guideline of the Johns Hopkins University, Baltimore, MD, USA; http://web.jhu.edu/animalcare/ procedures/mouse.html), i.e. about 80 mg genistein/kg mouse/day (Odle et al. 2017).

The animals were kept in the Department of Biomedical Research in macrolon cages 375 mm×215mm×150 mm at standard conditions (23□1° C., 55□10% humidity, 12 h day-night-cycle) and daily checked by the researcher and or animal facility staff. Chow and water were provided ad libitum.

### Urine Collection and Measurements

Spot urine was collected every week and Creatinine and protein levels were measured in the Clinical Institute for Laboratory Medicine of the Medical University Vienna.

Urinary albumin was measured by ELISA. 96-well plates were coated over night at 4° C. with 100 μl antibody solution Anti Mouse Serum Albumin antibody (Abcam ab34807, Lot GR3242102-4; diluted 1:2,000) in coating buffer (3.7g NaHCO₃; 0.64 g Na₂CO₃; 1L distilled water). The remaining procedure was performed at room temperature. Wells were washed 3× with HBSS (Sigma H8264-500ML) +0,05% Tween-20 (Bio-Rad, #1706531) and blocked in ELISA/ELISPOT Diluent (eBioscience, 00-4202-56) and washed again for 3×. Samples and standard curve were both diluted in ELISA/ELISPOT diluent were incubated for 2 h, followed by 3 wash steps with HBSS+0.05% Tween-20, incubated with HRP antibody Anti Mouse Serum Albumin antibody (Abcam ab19195, Lot GR3242102-4; diluted 1:100,000) for 1 h, followed by 5 was steps. Reaction was developed with TMB One Solution (Promega, G7431) and stopped with 2N H₂SO₄. Standard curves were generated with Mouse Albumin (Merck/Sigma, 126674-25MG). 450nm absorbance was measured on an Epoch Microplate reader (BioTek) using Gen5 1.10 software.

### Histology

At the end of the experiment kidneys were removed, embedded in paraffin and PAS (Periodic acid-Schiff) staining was performed in the Department of Clinical Pathology by expert research technicians. Histology was thoroughly evaluated by an expert nephro-pathologist and the amounts of sclerotic glomeruli and protein casts was quantified.

### Statistics

R version 4.0.2 was used for statistical analysis. Student's t-test was used for comparing UPCR and UACR values between samples from treated and control mice for each individual time point and to assess differences in histological parameters. Repeated measures ANOVA was performed to analyse the drug's impact on UPCR and UACR over time using the respective functions in the rstatix package (V0.6.0). ggplot2 (V3.3.2) and ggpubr (V0.4.0) packages were used for generating the graphical visualizations.

### Experimental Setup

18 Balb/c mice were injected with 10.5mg/kg adriamycin into the tail vein (see table 1). Two days (adriamycin nephropathy) post experiment start the active group received standard chow supplemented with 500 mg/kg genistein, while control mice stayed with standard chow. Spot urine was collected weekly, kidneys were harvested at sacrifice.

In the following Table 1, an overview of the mice that were used in the experiment is shown.

TABLE 1

Experiment 1

| | started | completed |
|---|---|---|
| Control | 9 | 9 |
| Genistein | 9 | 9 |

### Results

Genistein Significantly Reduces Proteinuria

The determination of the urinary protein to creatinine ratio (UPCR) revealed a marked amelioration after genistein therapy.

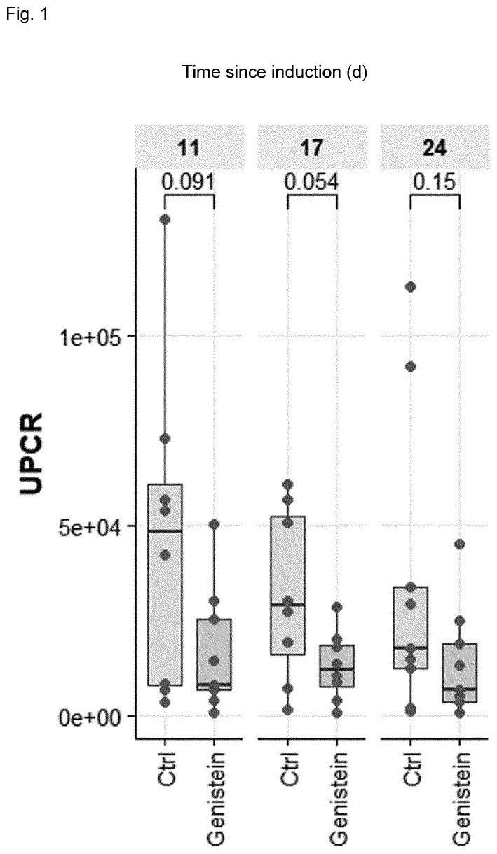

FIG. 1 depicts box plots and p-values of all individual collected urines. Repeated measures ANOVA revealed a significant beneficial impact of genistein treatment over time on outcome determined based on UPCR levels.

ANOVA Table (type III tests) - UPCR

| | Effect | DFn | DFd | F | p | p < .05 | ges |
|---|---|---|---|---|---|---|---|
| 1 | Experiment.drug | 1 | 60 | 10.357 | 0.002 | * | 0.147 |
| 2 | Timepoint.days | 3 | 60 | 3.631 | 0.018 | * | 0.154 |
| 3 | Experiment.drug:Timepoint.days | 3 | 60 | 0.978 | 0.409 | | 0.047 |

### REFERENCES

Bose B., Cattran D., Glomerular Diseases: FSGS, 2014, Clin. J. Am.Soc.Nephrol., 9, 626-632

Cayla G. et al., Flow cytometric assessment of vasodilator-stimulated phosphoprotein: prognostic value of recurrent cardiovascular events after acute coronary syndromes, 2008, 101(11-12), 743-51

Chen A. et al., Soluble RARRES1 induces podocyte apoptosis to promote glomerular disease progression, 2020, J.Clin.lnvest, 130(10), 5523-5535

Cheng WX, et al. Genistein inhibits angiogenesis developed during rheumatoid 10 arthritis through the IL-6/JAK2/STAT3NEGF signalling pathway, 2019, J Orthop Translat., 22, 92-100

Chung CF. et al., Intrinsic tumor necrosis factor-a pathway is activated in a subset of patients with focal segmental glomerulosclerosis, 2019, PLoS One, 14(5):e0216426

D'Agati V., Pathologic classification of focal segmental glomerulosclerosis, 2003, Semin Nephrol, 23(2), 117-34

Fioretto, P. et al., 1996, Patterns of renal injury in NIDDM patients with microalbuminuria, Diabetologia, vol. 39, no. 12, pp. 1569-1576

Gebeshuber C A, et al. Focal segmental glomerulosclerosis is induced by microRNA-193a and its downregulation of WT1, 2013, Nat Med., 19(4),481-487 Gholampour F, Mohammadi Z, Karimi Z, Owji S M. Protective effect of genistein in a rat model of ischemic acute kidney injury, 2020, Gene, 753, 144789

Harris J. J. et al., Active proteases in nephrotic plasma lead to a podocin-dependent phosphorylation of VASP in podocytes via protease activated receptor-1, 2013, J Pathol., 229(5), 660-71

Javanbakht M H, Sadria R, Djalali M, et al. Soy protein and genistein improves renal antioxidant status in experimental nephrotic syndrome. Revista Nefrologia. 2014, 34(4), 483-490.Jia Qiang et al., Genistein attenuates renal fibrosis in streptozotocin-induced diabetic rats. Molecular medicine reports, 2018, 19(1), 423-431.

Lloyd V. A. Jr., 2016, The Art, Science, and Technology of Pharmaceutical Compounding, 5th Edition, elSBN: 1-58212-263-6

Ma W, et al., Genistein alleviates 6-amyloid-induced inflammatory damage through regulating Toll-like receptor 4/nuclear factor κB, 2015, J Med Food., 18(3), 273-279.

Mentor-Marcel R, et al., Dietary genistein improves survival and reduces expression of osteopontin in the prostate of transgenic mice with prostatic adenocarcinoma (TRAMP), 2005, J Nutr., 135(5), 989-95

Palanisamy N, et al., Genistein modulates NF-κB-associated renal inflammation, fibrosis and podocyte abnormalities in fructose-fed rats, 2011, Eur J Pharmacol., 667(1-3), 355-364

Pedigo C. E. et al., Local TNF causes NFATc1-dependent cholesterol-mediated podocyte injury, 2016, J. Clin. Invest, 126(9), 3336-50

Qi C. et al., Classification and differential diagnosis of diabetic nephropathy, Journal of Diabetes Research, 2016, ID8637138, 1-7.

Rogacka D. et al., Regulation of podocytes function by AMP-activated protein kinase, 2020, Arch Biochem Biophys, 692, 108541

Rosenberg A. Z. and Kopp, J. B., Focal segmental glomerulosclerosis, Clin J Am Soc Nephrol 12: 502-517, 2017

Sakata T. et al., Antiplatelet therapy effectively reduces plasma plasminogen activator inhibitor-1 levels, 2011, Artherosclerosis, 214(2), 490-1

Santibariez J F, et al. Genistein and curcumin block TGF-beta 1-induced u-PA expression and migratory and invasive phenotype in mouse epidermal keratinocytes, 2000, Nutr Cancer., 37(1), 49-54.

Siller-Matula J. M. et al., Multiple electrode aggregometry predicts stent thrombosis better than the vasodilator-stimulated phosphoprotein phosphorylation assay, 2010, 8(2), 351-9

Song M, et al. Genistein exerts growth inhibition on human osteosarcoma MG-63 cells via PPARγ pathway, 2015, Int J Oncol., 46(3), 1131-40.

Sundaram M K, et al. Genistein Modulates Signaling Pathways and Targets Several Epigenetic Markers in HeLa Cells, 2019, Genes (Basel), 10(12), 955

Tervaert, T. W. C., et al., Pathologic classification of diabetic nephropathy, Journal of the American Society of Nephrology, 2010, vol.21, no.4, pp. 556-563

Tsai PY. et al., Antroquinonol reduces oxidative stress by enhancing the Nrf2 signaling pathway and inhibits inflammation and sclerosis in focal segmental glomerulosclerosis mice, 2011, Free Radic Biol Med, 50(11), 1503-16

Tong M, et al. Genistein attenuates advanced glycation end product-induced expression of fibronectin and connective tissue growth factor, 2012, Am J Nephrol., 36(1), 34-40

Troyanov S, et al.; Toronto Glomerulonephritis Registry Group. Focal and segmental glomerulosclerosis: definition and relevance of a partial remission, 2005, J Am Soc Nephrol., 16(4),1061-1068

Wang Y, et al. Genistein and Myd88 Activate Autophagy in High Glucose-Induced Renal Podocytes In Vitro, 2018, Med Sci Monit., 24:4823-4831

Yang S M., et al., Citral is renoprotective for focal segmental glomerulosclerosis by inhibiting oxidative stress and apoptosis and activating Nrf2 pathway in mice, 2013, PLoS One, 8(9), e74871

Yazdani Y, et al. Genistein Suppression of Matrix Metalloproteinase 2 (MMP-2) and Vascular Endothelial Growth Factor (VEGF) Expression in Mesenchymal Stem Cell Like Cells Isolated from High and Low Grade Gliomas, 2016, Asian Pac J Cancer Prey., 17(12), 5303-5307

Yousefinejad, A., et al., The Effect of Genistein, L-carnitine and their combination on gene expression of hepatocyte PPAR-a and CPT-1 in experimental nephrotic syndrome. Progress in Nutrition, 2019, 21(1-S), 163-172.

Zhang T, et al. Activation of nuclear factor erythroid 2-related factor 2 and PPARγ plays a role in the genistein-mediated attenuation of oxidative stress-induced endothelial cell injury, 2013 Br J Nutr., 109(2), 223-35

Zhou X, et al. Genistein antagonizes inflammatory damage induced by β-amyloid peptide in microglia through TLR4 and NF-κB, 2014 Nutrition., 30(1),90-5

1. A method of treating a non-diabetic subject suffering from or at risk of developing focal segmental glomerulosclerosis (FSGS) comprising the step of administering a pharmaceutically effective amount of a preparation comprising genistein, or a salt or solvate thereof, to the subject.

2. The method of claim 1, wherein genistein is administered as the sole active agent.

3. The method of claim 1, wherein the FSGS is a recurrent FSGS, primary FSGS, or native FSGS.

4. The method of claim 1, wherein the preparation is a medicinal product or a drug product comprising genistein and a pharmaceutically acceptable carrier.

5. The method of claim 1, wherein the preparation is administered systemically.

6. The method of claim 1, wherein the genistein in the preparation comprises the structure:

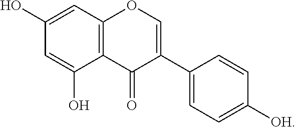 [SMILES: O=c1c(-c2ccc(O)cc2)coc2cc(O)cc(O)c12]

7. The method of claim 6, wherein the preparation comprises about 10 to 100 grams of genistein.

8. The method of claim 1, wherein said the preparation is formulated for intravenous, intramuscular, subcutaneous, intradermal, transdermal, or oral administration.

9. The method of claim 1, wherein said preparation is administered to the subject as a spray, a powder, a gel, an ointment, a cream, a foam, a liquid solution, a lotion, a gargle solution, an aerosolized powder, an aerosolized liquid formulation, granules, capsules, drops, a tablet, syrup, a lozenge, or a preparation for infusion or injection.

10. The method of claim 1, wherein the subject is at risk of developing FSGS.

11. The method of claim 1, wherein the preparation is combined with a further preparation having one or more additional active agents.

12. The method of claim 11, wherein the additional active agents are selected from the group consisting of antiviral drugs, anticoagulants, immune modulators, antibody preparations from human sources, monoclonal antibodies, intensive care medications, antihypertensive agents, statins, vasodilators, steroids, cytotoxic drugs, diuretics, non-steroidal anti-inflammatory drugs (NSAIDs), cholesterol or triglyceride reducing agents, and PPAR agonists.

13. The method of claim 3, wherein the FSGS is a glomerulopathy characterized by any one of heavy proteinuria, nephrotic syndrome, glomerulonephritis, membranoproliferative glomerulonephritis, IgA glomerulonephritis, progressive renal failure, or glomerular lesions on histopathology.
